(19) United States
(12) Patent Application Publication
(10) Pub. No.: US 2005/0176812 A1
(43) Pub. Date: Aug. 11, 2005

(54) Method of treating cancer

(76) Inventors: Pamela Cohen, Tenafly, NJ (US); Christopher Jagoe, Chatham, NJ (US); Frederick Ray Kinder JR., Boston, MA (US); Peter Lassota, Westborough, MA (US); Helmut Thomas, Heitersheim (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/981,336

(22) Filed: Nov. 4, 2004

Related U.S. Application Data
(60) Provisional application No. 60/517,897, filed on Nov. 6, 2003.
(60) Provisional application No. 60/572,903, filed on May 20, 2004.

(51) Int. Cl. A61K 31/366

(52) U.S. Cl. 514/460

(57) ABSTRACT

This invention relates to an alternative regimen for the administration of a discodermolide that is useful for the treatment of cancer. According to the inventive regimen the human patient receives a dose of the discodermolide only once every 3 weeks over the period that the treatment is carried out.

The present invention relates to a superior treatment regimen for the administration of a discodermolide to a human subject for the treatment of cancer, particularly tumorous cancers, and other pathological conditions that are treatable with a microtubule-stabilizing agent. The inventive treatment regimen proposes to offer benefit by maintaining balancing efficacy and toxicity of the discodermolide.

Discodermolides useful for treating tumor diseases and other conditions are disclosed in U.S. Pat. Nos. 5,010,099; 4,939,168; 5,840,750; and 5,681,847, which are here incorporated by reference in their entirety. The latter patents disclose dosage levels of the administered active ingredients can be intravenous (i.v.), 0.01 mg/kg to about 20 mg/kg; intraperitoneal, 0.01 mg/kg to about 100 mg/kg; subcutaneous, 0.01 mg/kg to about 100 mg/kg; intramuscular, 0.01 mg/kg to about 100 mg/kg; orally, 0.01 mg/kg to about 200 mg/kg, preferably about 1-100 mg/kg; intranasal instillation, 0.01 mg/kg to about 20 mg/kg; and aerosol, 0.01 mg/kg to about 20 mg/kg of animal (body) weight. However, this disclosure only suggests an appropriate daily dose and does not suggest that the discodermolide should be administered on a 3-week dosing schedule, i.e., treatment every 3 weeks or every 21 days.

The present invention is based on the discovery that when a discodermolide is administered to a human on a 3-week dosing schedule as described herein, there is a balance of efficacy and toxicity. More frequent dosing of the discodermolide could result in the toxicity of the compound outweighing the benefits of the treatment.

According to the present invention, discodermolide is administered to the human subject less frequently than on a daily basis. In particular, this aspect of the present invention relates to a treatment regimen whereby the discodermolide is administered on a dosing schedule of at least once every 3 weeks.

As used herein, the expression “week” means 7 consecutive days. Thus, a 3-week period is 21 consecutive days starting on any day of the calendar week. Administration once “every 3 weeks” means that a unit dosage of the discodermolide is administered at least once followed by a 3-week period where the discodermolide is not administered, i.e., one time during a 22-day period, preferably on the same day during each 3-week period. The period where the discodermolide is not administered, also known as the “rest period” is the interval or number of days from administration of 1 complete dose of the discodermolide to the next administration of one complete dose of the active compound. For example, if the first complete dose of discodermolide is administered on day 1, the next dose will be administered after a rest period of 21 days later, or on day 22. This cycle is repeated for from 1 to several cycles, e.g., from 3 or 4 to 8 or more cycles.

In a first aspect, the present invention relates to a method of treating a human subject with a discodermolide, which comprises administering discodermolide to the human subject in a dosing periodicity ranging from about once every 3 weeks.

More specifically, this aspect of the present invention relates to a method of treating a human subject with a discodermolide, which comprises administering the discodermolide to the human subject followed by a rest period of 3 weeks or 21 days from the last administered dose. This cycle is repeated from 1-3 or more cycles.

In another aspect, the present invention relates to a method for administering discodermolide to a human subject, which comprises administering a pharmaceutically effective amount of discodermolide to the human subject at least once followed by 3 weeks before administration of the next dose.

Thus, the present invention relates a method for administering discodermolide to a human subject, which comprises administering a pharmaceutically effective amount of the discodermolide to the human subject at least every 3 weeks.

The discodermolide is advantageously administered to the human subject by i.v. infusion at a pharmaceutically effective dosage in the range of from about 0.01-100 mg/m² on days when the discodermolide is administered.

Specifically, the inventive method relates to the above described methods of administering discodermolide to a human subject wherein the pharmaceutically effective dose of discodermolide when administered, is in the range from about 0.1-75 mg/m² doses, preferably about 0.5-60 mg/m² or about 0.6-20 mg/m². In some embodiments the dose may be 14.4 mg/m², between 0.6-9.6 mg/m² or 19 mg/m² and above, such as 25 mg/m².

Preferably the concentration and dosage strength may be such to achieve an effective dose level of about 0.5-70 mg every 3 weeks, more preferably 1-30 mg every 3 weeks, or more preferably 30-40 mg every 3 weeks. The dose received by i.v. administration and the blood concentration may be determined accurately on the basis of known in vivo and in vitro techniques.

The pharmaceutical formulation of discodermolide comprises an infusion concentrate of discodermolide and a pharmaceutically acceptable organic solvent, such as an alcohol, preferably propylene glycol. The discodermolide may be present in an infusion concentrate in a concentration of 0.1-50 mg/mL, e.g., 1-50 mg/mL or 0.5-50 mg/mL, more preferably 0.1-20 mg/mL, or 0.3-5 mg/mL or 0.5-4 mg/mL, 0.6-3 mg/mL, or 2 mg/mL.

The infusion concentrates may be diluted in a diluent vehicle comprising a pharmaceutically acceptable organic solvent in saline. Examples of the organic solvent include propylene glycol, ethanol, Tween 80, benzoic acid, benzyl alcohol and mixtures thereof. Examples of diluent vehicles include ethanol in normal saline; ethanol and Tween 80 in normal saline; ethanol/benzoic acid in normal saline; and ethanol/benzoic acid/benzyl alcohol in normal saline. The ratio of organic solvent to saline will be such to obtain suitable solubility of discodermolide, however the amount of organic solvent is limited by practical limitations. If the ratio of organic solvent is too low, discodermolide is not soluble. However, the amount of organic solvent cannot be so high that too much organic solvent is administered to a patient. Preferred is 10-20% w/v ethanol in saline or 15-17% w/v ethanol in saline. The most preferred diluent vehicle comprises 16.3% w/v ethanol in saline, where each mL contains 7.3 mg NaCl and 163 mg ethanol.

The amount of diluent used in admixture with the infusion concentrate in order to form an infusion solution may be chosen according to the desired concentration of discodermolide in the infusion solution. Preferably the infusion solution is prepared by mixing a vial or ampoule of infusion concentrate aforementioned with a diluent vehicle.

The infusion concentrate and diluent vehicle are prepared and stored separately. Prior to administration the infusion concentrate and diluent vehicle are combined to form an infusion solution. The infusion solution so formed may be preferably used immediately or within a short time of being formed, e.g., within 8 hours. Alternatively, the infusion concentrate and a predetermined amount of diluent, may be loaded each into separate chambers of a double-chamber vial system and only mixed immediately prior to i.v. administration to a patient.

The amount of diluent used in admixture with the infusion concentrate to form an infusion solution may be chosen so as to obtain a desired concentration of discodermolide in the infusion solution. The amount of diluent used is also chosen so that the solution is stable long enough to be administered. Concentration of infusion solutions are 0.1-2.0 mg/mL, or 0.1-1.5 mg/mL or preferably 0.5-8.0 mg/mL or 0.77 mg/mL. Such solutions are found to be stable for up to 8 hours.

Preferably the infusion solution of the present invention is prepared by mixing the infusion concentrate with a diluent, such as 16.3% W/v ethanol in saline in a suitable container. A preferred formulation comprises 1 mL of 2 mg/mL infusion concentrate diluted with 1.6 mL of 16.3% w/v ethanol in saline to yield an infusion solution containing 0.77 mg/mL discodermolide in 40% propylene glycol, 10% ethanol and 50% normal saline. This solution is administered via iv infusion, or push into Y tubing containing a 0.9% normal saline drip, directly into the vein and the drug solution residue in the tubing will be flushed with normal saline. The volume of discodermolide solution given via i.v. push will vary from 0.3 mL (at the starting does of 0.2 mg or 0.13 mg/m²) to 26 mL (for a dose of 20 mg or 11.43 mg/m²) or 43 mL (for a dose of 33 mg or 19.2 mg/m²). It has been found that direct i.v. infusion into the vein followed by a normal saline flush will not cause any precipitation of discodermolide.

Preferably the concentration of the infusion solution and dosage strength may be such to achieve an effective dose level of about 0.5-70 mg every 3 weeks, more preferably 1-30 mg every 3 weeks, or more preferably 30-40 mg every 3 weeks. The dose received by i.v. administration and the blood concentration may be determined accurately on the basis of known in vivo and in vitro techniques.

The utility of infusion solutions of the present invention may be observed in standard clinical trials in, e.g., known indications of discodermolide dosages giving equivalent blood levels of discodermolide, e.g., using dosages in the range of about 0.1-75 mg/m2 of discodermolide, preferably 0.5-60 mg/m² or 0.6-25 mg/m² for daily, weekly, every 2 weeks or every 3 weeks administration for a 75 kg mammal, e.g., an adult human of 1.73 m², and in standard animal models. Preferably, the dosage is from about 0.1-30 mg/m². Examples of preferred doses include 0.6 mg/m², 1.2 mg/m², 2.4 mg/m², 4.8 mg/m², 9.6 mg/m², 14.4 mg/m², 19.2 mg/m² and 25 mg/m². The preferred dose is 1-40 mg or 30-40 mg once every 3 weeks. This dose is administered to a human by i.v. administration.

The inventive method especially relates to the above described methods of administering discodermolide to a human subject to treat a tumor condition, especially cancer. Examples of cancers and tumors are disclosed in U.S. Pat. Nos. 5,010,099; 4,939,168; 5,840,750; 5,681,847; and WO 01/74355, which are here incorporated by reference in their entirety.

The inventive dosage regimen applies to the use of discodermolide, alone, or as part of a combination treatment therapy wherein it is co-administered with 1 or more additional pharmaceutical products useful for treating tumors, especially cancerous tumors. For purposes of this application co-administered means that the patient is treated with both drugs according to the proper schedule for each, but not necessarily that both drugs are administered together at the same time. Thus, the discodermolide may be administered alone or in combination with other anticancer agents in accordance with the present inventive dosage regimen.

The discodermolide is administered to the subject by methods known in the art for administering pharmaceutical products, e.g., orally, rectally or parenterally, preferably as an i.v. infusion.

1. A method for administering discodermolide to a human subject, which comprises a dosing regimen comprising administering a pharmaceutically effective dose of the discodermolide to the human subject at least once followed by a 3-week time period before administration of the next dose.

2. A method of claim 1, wherein the dosage regimen is repeated for 1 or more cycles.

3. A method of claim 1, wherein the pharmaceutically effective dose is in the range from about 0.01 mg/m² to about 75 mg/m².

4. A method of claim 3, wherein the pharmaceutically effective dose is in the range from about 0.1 mg/m² to, about 70 mg/m².

5. A method of claim 3, wherein the pharmaceutically effective dose is in the range from about 0.5 mg/m² to about 60 mg/m².

6. A method of claim 1, wherein the discodermolide is administered to treat a tumor condition.

7. A method of claim 1, wherein the discodermolide is administered as part of a combination treatment therapy with 1 or more additional pharmaceutical products useful for treating tumors.

8. A method for administering discodermolide to a human subject, which comprises administering a pharmaceutically effective amount of the discodermolide, to the human subject once every 3 weeks for a period of 3 weeks or longer.

9. A method of claim 8, wherein the weekly dosage regimen is repeated for 1 or more cycles.

10. A method of claim 8, wherein the pharmaceutically effective dose is in the range from about 0.01 mg/m² to about 100 mg/m².

11. A method of claim 10, wherein the pharmaceutically effective dose is in the range from about 0.1 mg/m² to about 75 mg/m².

12. A method of claim 11, wherein the pharmaceutically effective dose is in the range from about 0.5 mg/m² to about 60 mg/m².

13. A method of claim 8, wherein the discodermolide is administered to treat a tumor condition.

14. A method of claim 13, wherein the discodermolide is administered as part of a combination treatment therapy with 1 or more additional pharmaceutical products useful for treating tumors.

15. A method for administering discodermolide to a human subject, which comprises administering a pharmaceutically effective amount of the discodermolide to the human followed by a period of 3 weeks wherein the discodermolide is not administered and this schedule is followed for 1 or more cycles.

16. A method of claim 15, wherein the discodermolide is administered as part of a combination treatment therapy with 1 or more additional pharmaceutical products useful for treating tumors.
